(19) United States
(12) Patent Application Publication
(10) Pub. No.: US 2020/0000692 A1
(43) Pub. Date: Jan. 2, 2020

(54) Cold Process Emulsifier Compositions

(71) Applicant: Hallstar Beauty and Personal Care Innovations Company, Chicago, IL (US)

(72) Inventors: Eva Baldaro, Milan (IT); Vincenzo Placa, Polizzi Generosa (IT); Mattia Pozzi, Monticello Brianza (IT); Shengkui Hu, Darien, IL (US)

(21) Appl. No.: 16/566,439

(22) Filed: Sep. 10, 2019

Related U.S. Application Data
(63) Continuation of application No. PCT/US2017/021943, filed on Mar. 10, 2017.

(51) Int. Cl. A61K 8/06; A61K 9/107; A61K 8/81; A61K 8/34; A61K 8/92; A61K 8/73; A61K 8/44; A61Q 19/00; A61Q 5/00

(52) U.S. Cl. CPC A61K 8/062; A61K 9/107; A61K 8/8147; A61K 8/345; A61Q 5/00; A61K 8/73; A61K 8/44; A61Q 19/00; A61K 8/922

(57) ABSTRACT

The present disclosure relates, according to some embodiments, to a cold process emulsifier composition comprising: at least one acrylic polymer, wherein the at least one acrylic polymer is neutralized after polymerization by at least one basic solution; a hydrophobic continuous phase comprising at least one solvent, wherein the at least one solvent has a polarity index from about 3 millinewtons per meter (mN/m) to about 30 mN/m; a hydrophilic discontinuous phase comprising water; at least one emulsifier, wherein the at least one emulsifier has a hydrophilic-lipophilic balance from about 2 to about 5; and at least one inverting agent, wherein the at least one inverting agent has a hydrophilic-lipophilic balance of at least about 10; wherein the cold process emulsifier composition is configured to form a cold process oil in water emulsion when the cold process emulsifier composition is contacted with a hydrophilic continuous phase comprising at least one aqueous solvent.

### CROSS-REFERENCE TO RELATED APPLICATIONS

This application is a continuation application of International Application No. PCT/US2017/021943 filed Mar. 10, 2017, the contents of which is hereby expressly incorporated by reference in its entirety, including the contents and teachings of any references contained therein.

### FIELD OF THE DISCLOSURE

The present disclosure relates, in some embodiments, to cold process emulsifier compositions including, methods of preparing cold process emulsifier compositions and cold process oil in water emulsions, and use of such compositions to formulate personal care products (e.g., sun care product, skin care, toiletries), pharmaceuticals, and cosmetic compositions.

### BACKGROUND OF THE DISCLOSURE

Emulsions, or colloids composed of mixtures of two or more liquids that are typically immiscible, may be employed in the food, pharmaceutical, cosmetic, personal care product, chemical, culinary, dermatological, medical, recreational, construction, polymer, textile, automotive, and agriculture industries. Currently practiced methods of producing and stabilizing oil in water and/or water in oil emulsions involve high temperatures and high rates of mixing, specialized equipment to handle high temperature and high rates of mixing, and produce formulations in which emulsion stability typically breaks down through mechanisms comprising flocculation, creaming, and coalescence. Solid emulsifiers (e.g., surfactants) and detergents can be added to emulsions to increase the thermodynamic stability of said emulsion, but many of these surfactants and/or detergents are not suitable for formulating personal care product, pharmaceutical, and cosmetic compositions. Also, many emulsions involve petrochemical and/or synthetic oil-phase solvents that are not renewable resources. Besides contributing to negative reactions like poor taste, undesirable feel, dermatitis, and toxicity, these hazardous chemicals increase the environmental impact associated with the development, use, and product disposal.

### SUMMARY

Accordingly, a need has arisen for improved cold process emulsifier compositions that may not need to be heated or combined with synthetic solvents, surfactants, and/or detergents to be formed and/or to remain stable emulsions.

According to some embodiments, the present disclosure relates to a cold process emulsifier composition comprising an acrylic polymer neutralized after polymerization by a basic solution; a hydrophobic continuous phase comprising a solvent, wherein the solvent may have a polarity index (e.g., from about 3 millinewtons per meter (mN/m) to about 30 mN/m); a hydrophilic discontinuous phase comprising water; an emulsifier, wherein the emulsifier may have a desired hydrophilic-lipophilic balance (e.g., from about 2 to about 5); and an inverting agent, wherein the inverting agent may have a hydrophilic-lipophilic balance (e.g., of at least about 10); wherein the cold process emulsifier composition may be configured to form a cold process oil in water emulsion when the cold process emulsifier composition may be contacted with a hydrophilic continuous phase comprising an aqueous solvent.

In some embodiments, the present disclosure relates to a method of making a cold process oil in water emulsion, the method comprising: (a) combining an aqueous discontinuous phase with a cold process emulsifier component to form a cold process emulsifier composition, a cold process emulsifier component comprising: an acrylic polymer neutralized after polymerization by a basic solution; a hydrophobic continuous phase comprising a solvent, wherein a solvent may have a polarity index from about 3 millinewtons per meter (mN/m) to about 30 mN/m; an emulsifier, wherein the emulsifier may have a hydrophilic-lipophilic balance (HBL) from about 2 to about 5; and an inverting agent, wherein the inverting agent may have a hydrophilic-lipophilic balance of at least about 10; (b) mixing the cold process emulsifier composition form a mixed cold process emulsifier composition; and (c) combining the mixed cold process emulsifier composition with a hydrophilic continuous phase to form the cold process oil in water emulsion. A method of making a cold process oil in water emulsion may comprise formulating a product with the cold process oil in water emulsion at a temperature from about 5° C. to about 40° C., wherein the product comprises personal care products, pharmaceutical compositions, and cosmetic compositions.

According to some embodiments, a method of making a cold process emulsifier composition, the method comprising: (a) combining an acrylic polymer with at least one emulsifier and a hydrophobic continuous phase comprising a solvent to form an acrylic polymer continuous phase mixture; (b) combining the acrylic polymer continuous phase mixture with a hydrophilic discontinuous phase and a basic solution to form a neutralized polyacrylate mixture; and (c) combining an inverting agent with the acrylic polymer continuous phase mixture to form the cold process emulsifier composition. A method of making a cold process emulsifier composition may comprise formulating a product with the cold process emulsifier composition at a temperature from about from about 5° C. to about 40° C., wherein the product comprises personal care products, pharmaceutical compositions, and cosmetic compositions.

In some embodiments, a basic solution comprises water and a base selected from the group consisting of lithium hydroxide, sodium hydroxide, potassium hydroxide, cesium hydroxide, calcium hydroxide, ammonium hydroxide, potassium bicarbonate, sodium bicarbonate, calcium bicarbonate, ammonium bicarbonate, potassium carbonate, sodium carbonate, calcium carbonate, ammonium carbonate. A basic solution may have a concentration of a base from about 5 wt. % to about 70 wt. %, by weight of a basic solution. An inverting agent may be selected from the group consisting of polyglyceryl olivate and polyglyceryl oleate. An inverting agent may be selected from the group consisting of naturally derived surfactants such as acyl glucosides, acyl glutamates, acyl sarcosinates, and acyl polyglyceryl esters. A inverting agent may be polyglyceryl olivate. An inverting agent may be present at a concentration from about 0 wt. % to about 10 wt. %, or about 1 wt. % to about 8 wt. %, or about 2 wt. % to about 6 wt. %, about 3 wt. % to about 4 wt. %, by weight of the cold process emulsifier composition.

An acrylic polymer may be a homopolymer or copolymer of monomers selected from the group consisting of acrylic acid, methacrylic acid, ethyl acrylic acid, propyl acrylic acid, butyl acrylic acid, methacrylate, ethyl acrylate, propyl acrylate, butyl acrylate, hydroxyethyl acrylate, C₁₀₋₃₀ alkyl acrylate, methyl methacrylate, ethyl methacrylate, propyl methacrylate, butyl methacrylate, hydroxyethyl methacrylate, C₁₀₋₃₀ alkyl methacrylate, acrylonitrile, methyl acrylonitrile, ethyl acrylonitrile, propyl acrylonitrile, butyl acrylonitrile, hydroxyethyl acrylonitrile, taurate, and C₁₀₋₃₀ alkyl acrylonitrile. An acrylic polymer may be present at a concentration from about 5 wt. % to about 50 wt. %, by weight of the cold process emulsifier composition. A solvent may be selected from the group consisting of ethylhexyl oleate, ethylhexyl olivate, ethyl olivate, ethyl hexyl linoleate, ethyl hexyl linolenate, ethyl hexyl palmitate, ethyl hexyl stearate, oleic acid, linoleic acid, linolenic acid, palmitic acid, and stearic acid. A solvent may be selected from triacylglycerols and glycerol ethers formed from glycerol and fatty acids selected from the group consisting of oleic acid, linoleic acid, linolenic, palmitic acid, stearic acid, and linolenic acid. A solvent may be present at a concentration from about 20 wt. % to about 80 wt. %, by weight of the cold process emulsifier composition.

According to some embodiments, an emulsifier may be selected from the group consisting of sorbitan olivate, sorbitan laurate, sorbitan stearate, sorbitan linoleate, sorbitan myristate, sorbitan palmitate, sorbitan arachidonate, cetearyl olivate, cetearyl laurate, cetearyl stearate, cetearyl linoleate, cetearyl myristate, cetearyl palmitate, cetearyl arachidonate, polyglyceryl olivate, polyglyceryl laurate, polyglyceryl stearate, polyglyceryl linoleate, polyglyceryl myristate, polyglyceryl palmitate, and polyglyceryl arachidonate. An emulsifier may be present at a concentration from about 1 wt. % to about 20 wt. %, by weight of the cold process emulsifier composition. An aqueous solvent may be selected from the group consisting of water, water-based solutions, aqueous-based solutions, aqueous-based solutions comprising glycerin, aqueous-based solutions comprising ethylene and/or propylene glycols, and brine.

In some embodiments, a cold process emulsifier composition may have a viscosity of about 5000 cP to about 25000 cP. A cold process oil in water emulsion may be suitable for formulating personal care products, pharmaceutical compositions, and cosmetic compositions. A solvent may have a polarity index from about 3 millinewtons per meter (mN/m) to about 30 mN/m. A hydrophilic discontinuous phase comprises water. A neutralized polyacrylate mixture may have a pH from about 6 to about 8. An emulsifier may have a hydrophilic-lipophilic balance from about 2 to about 5. An inverting agent may have a hydrophilic-lipophilic balance of at least about 10. Combining the aqueous discontinuous phase with a cold process emulsifier component to form a cold process emulsifier composition may reach a concentration of an inverting agent from about 1 wt. % to about 10 wt. %, by weight of the cold process emulsifier composition. A cold process emulsifier composition comprises a viscosity of about 5000 cP to about 25000 cP. A cold process oil in water emulsion may be suitable for formulating personal care products, pharmaceutical compositions, and cosmetic compositions.

According to some embodiments, the present disclosure relates to a method of formulating a cold process emulsion product, the method comprising mixing a cold process emulsion composition with at least one emulsion product additive, wherein the cold process emulsion composition comprises: at least one acrylic polymer neutralized after polymerization by at least one basic solution; a hydrophobic continuous phase comprising at least one solvent, wherein the at least one solvent has a polarity index from about 3 millinewtons per meter (mN/m) to about 30 mN/m; a hydrophilic discontinuous phase comprising water; at least one emulsifier, wherein the at least one emulsifier has a hydrophilic-lipophilic balance from about 2 to about 5; and at least one inverting agent, wherein the at least one inverting agent has a hydrophilic-lipophilic balance of at least about 10. A cold process emulsion product may comprise personal care products, pharmaceutical compositions, cosmetic compositions, or combinations thereof. At least one emulsion product additive comprises water, glycerin, propylene glycol, butylene glycol, or combinations thereof.

### DETAILED DESCRIPTION

The present disclosure relates, in some embodiments, to cold process emulsifier compositions, methods of making cold process emulsifier compositions, and methods of making cold process oil in water emulsions. Cold process emulsifier compositions and cold process oil in water emulsions may be used to formulate personal care products, pharmaceuticals, and cosmetic compositions, wherein formulation occurs at about room temperature (i.e., about 20° C. to about 25° C.). An emulsion may comprise a mixture of two liquids that, once mixed, may separate over a time duration. For example, in an emulsion, one of the liquids may be a hydrophobic, oil based material (e.g., olive oil) and the other liquid may be a hydrophilic, aqueous based material (e.g., water). Since emulsions may not be thermodynamically stable indefinitely and may separate and/or degrade over time (e.g., flocculation, creaming, and coalescence), emulsifiers (e.g., surfactants) may be employed to stabilize or further stabilize such emulsions over a duration of time and/or at a range of temperatures. A water in oil emulsion may have less water in proportion to oil, whereas an oil in water emulsion may have more oil in proportion to water.

Cold process emulsifier compositions may be included in, for example, personal care compositions (e.g., sun care product), pharmaceutical compositions, cosmetic compositions, dermatological compositions, culinary compositions, medical compositions, industrial compositions, and/or recreational compositions. Emulsions comprising petrochemical based oils (e.g., mineral oil) and harsh surfactants (e.g., sodium laureth sulfate) as stabilizers that may have undesirable qualities if included in compositions that are intended for humans (e.g., personal care products, pharmaceutical compositions, and/or cosmetic compositions compositions). In some embodiments, the present disclosure relates to cold process emulsifier compositions comprising an acrylic polymer neutralized in naturally derived plant based oils, naturally derived emulsifiers with low (e.g., from about 2 to about 7) and/or high (e.g., from about 8 to about 12) hydrophilic-lipophilic balances, and naturally derived plant based inverting agents with high hydrophilic-lipophilic balances. In some embodiments, compositions comprising naturally derived oils, emulsifiers, and/or inverting agents may be substantially free (e.g., free) of irritation, poor taste, toxicity and/or other side-effects that may result from contact with formulations containing petrochemical based oils and/or harsh surfactants. Further, cold process emulsifier compositions comprising naturally derived oils, emulsifiers, and/or inverting agents may desirably be able to be formed at lower than about 80° C., especially below 60° C., while remaining stable at a temperature of about 5 to about 40° C. for at least about 1 month, or at least about 2 months, or at least about 3 months, or at least about 4 months, or at least about 5 months, or at least about 6 months. Cold process emulsifier compositions comprising naturally derived oils, emulsifier, and/or inverting agents may desirably be useful in formulating personal care products, pharmaceuticals, and cosmetic compositions, wherein formulation occurs at a temperature of from about 5° C. to about 40° C. In some embodiments, lower temperatures may preserve the natural and/or intrinsic benefits of natural ingredients (e.g., naturally derived oils, emulsifiers, and/or inverting agents).

### Cold Process Emulsifier Compositions

According to some embodiments, the present disclosure relates to cold process emulsifier compositions. A cold process emulsifier composition may comprise at least one acrylic polymer, wherein the at least one acrylic polymer is neutralized after polymerization by at least one basic solution; a hydrophobic continuous phase comprising at least one solvent, wherein the at least one solvent has a polarity index from about 3 millinewtons per meter (mN/m) to about 30 mN/m; a hydrophilic phase comprising water; at least one emulsifier, wherein the at least one emulsifier has a hydrophilic-lipophilic balance from about 2 to about 7 (e.g., low hydrophilic-lipophilic balance); and optionally, at least one inverting agent, wherein the at least one inverting agent has a hydrophilic-lipophilic balance of at least about 10; wherein the cold process emulsifier composition is configured to form a cold process oil in water emulsion when the cold process emulsifier composition is contacted with a hydrophilic continuous phase comprising at least one aqueous solvent, and wherein the cold process emulsifier composition is configured to be formed at a temperature of less than about 60° C. At least one inverting agent may have a hydrophilic-lipophilic balance from about 8 to about 12 (e.g., high hydrophilic-lipophilic balance).

According to some embodiments, a cold process emulsifier composition may be formed at a temperature, for example, from about 5° C. to about 80° C. A cold process emulsifier composition may be formed at a temperature of less than about 5° C., or about 10° C., or about 15° C., or about 20° C., or about 25° C., or about 30° C., or about 35° C., or about 40° C., or about 45° C., or about 50° C., or about 55° C., or about 60° C., or about 65° C., or about 70° C., or about 75° C., or about 80° C.

According to some embodiments, a cold process emulsifier composition may be used to formulate a product at a temperature, wherein the temperature is from about 0° C. to about 80° C. A cold process emulsifier composition may be used at a temperature of less than about 5° C., or about 10° C., or about 15° C., or about 20° C., or about 25° C., or about 30° C., or about 35° C., or about 40° C., or about 45° C., or about 50° C., or about 55° C., or about 60° C., or about 65° C., or about 70° C., or about 75° C., or about 80° C. A cold process emulsifier composition may be used to formulate a product at a temperature from about 20° C. to about 25° C. A product may comprise a personal care product (e.g., sun care product, skincare, toiletries), a pharmaceutical, and a cosmetic composition. A cold process emulsifier composition may be used to formulate a product without heating or cooling the product during a formulation process.

In some embodiments, a cold process emulsifier composition may comprise at least one acrylic polymer, wherein the at least one acrylic polymer is neutralized after polymerization by at least one basic solution. Neutralization may be achieved when a composition comprising at least one acrylic polymer has a pH from about 4 to about 9. Neutralization may be achieved when a composition comprising at least one acrylic polymer has a pH from about 5 to about 8. Neutralization may be achieved when a composition comprising at least one acrylic polymer has a pH from about 6 to about 8. Neutralization may be achieved when a composition comprising at least one acrylic polymer has a pH from about 6.5 to about 7.5. Neutralization may be achieved when a composition comprising at least one acrylic polymer has a pH of about 7. Neutralization may be achieved when a composition comprising at least one acrylic polymer has a pH of about 4, or of about 4.5, or of about 5, or of about 5.5, or of about 6, or of about 6.5, or of about 7, or of about 7.5, or of about 8, or of about 8.5, or of about 9. A pH of a cold process emulsifier composition may be measured by diluting the cold process emulsifier composition to make an about 1 wt. % in water composition, and then measuring the pH of the 1 wt. % in water composition. For example, about 1 gram of a cold process emulsifier composition may be combined with about 99 g of water (e.g., demineralized water) to form a diluted cold process emulsifier composition, wherein the pH of the diluted cold process emulsifier composition may be measured.

In some embodiments, at least one basic solution may comprise water and at least one base selected from the group consisting of lithium hydroxide, sodium hydroxide, potassium hydroxide, cesium hydroxide, calcium hydroxide, ammonium hydroxide, potassium bicarbonate, sodium bicarbonate, calcium bicarbonate, ammonium bicarbonate, potassium carbonate, sodium carbonate, calcium carbonate, ammonium carbonate.

At least one base may be present in at least one basic solution at a concentration from about 5 wt. % to about 70 wt. %, by weight of the at least one basic solution. According to some embodiments, at least one base may be present in at least one basic solution at a concentration of about 5 wt. %, or about 10 wt. %, or about 15 wt. %, or about 20 wt. %, or about 25 wt. %, or about 30 wt. %, or about 35 wt. %, or about 40 wt. %, or about 50 wt. %, or about 55 wt. %, or about 60 wt. %, or about 65 wt. %, or about 70 wt. %, in each case, by weight of the at least one basic solution. At least one base may be present in at least one basic solution at a concentration of about 30 wt. %, by weight of the at least one basic solution. At least one base may be present in at least one basic solution at a concentration from about 5 wt. % to about 10 wt. %, or from about 10 wt. % to about 15 wt. %, or from about 15 wt. % to about 20 wt. %, or from about 20 wt. % to about 25 wt. %, or from about 25 wt. % to about 30 wt. %, or from about 35 wt. % to about 40 wt. %, or from about 40 wt. % to about 45 wt. %, or from about 45 wt. % to about 50 wt. %, or from about 50 wt. % to about 55 wt. %, or from about 55 wt. % to about 60 wt. %, or from about 60 wt. % to about 65 wt. %, or from about 65 wt. % to about 70 wt. %, in each case, by weight of the at least one basic solution. At least one base may be present in at least one basic solution at a concentration of about 5 wt. %, or about 10 wt. %, or about 15 wt. %, or about 20 wt. %, or about 25 wt. %, or about 30 wt. %, or about 35 wt. %, or about 40 wt. %, or about 50 wt. %, or about 55 wt. %, or about 60 wt. %, or about 65 wt. %, or about 70 wt. %, in each case, by weight of the at least one basic solution. At least one base may be present in at least one basic solution at a concentration from about 25 wt. % to about 35 wt. %, by weight of the at least one basic solution.

At least one acrylic polymer may be neutralized after polymerization while dispersed in a hydrophobic continuous phase (e.g., ethylhexyl olivate). At least one acrylic polymer may be neutralized after polymerization while dispersed in a hydrophilic continuous phase (e.g., water). At least one acrylic polymer may be neutralized after polymerization in presence of the at least one emulsifier, wherein the at least one emulsifier has a hydrophilic-lipophilic balance from about 2 to about 7. A neutralization step may occur at a temperature from about −15° C. to about 80° C., or about 0° C. to about 70° C., or about 5° C. to 60° C. A neutralizing agent may be added under stirring, which may keep a polymer well dispersed within the oil phase. A neutralizing agent may be added stepwise or continuously. In some embodiments, a working formulation of a neutralizing agent may have a concentration from about 10% to about 30% (w/w) of the agent in water (e.g., demineralized water).

Performing a neutralization step after the polymerization may permit using at least one high polarity hydrophobic phase and/or highly sensitive oils like esters or unsaturated fatty phases, that otherwise may not be compatible with the polymerization process and additives. A high polarity hydrophobic phase may have a polarity index of below about 30 mN/m. A neutralization step performed before forming a final composition with the polymer in a personal care formulation may reduce the time and complexity of oil in water compositions.

At least one acrylic polymer may be a homopolymer or copolymer of monomers selected from the group consisting of acrylic acid, methacrylic acid, ethyl acrylic acid, propyl acrylic acid, butyl acrylic acid, methacrylate, ethyl acrylate, propyl acrylate, butyl acrylate, hydroxyethyl acrylate, C₁₀₋₃₀ alkyl acrylate, methyl methacrylate, ethyl methacrylate, propyl methacrylate, butyl methacrylate, hydroxyethyl methacrylate, C₁₀₋₃₀ alkyl methacrylate, acrylonitrile, methyl acrylonitrile, ethyl acrylonitrile, propyl acrylonitrile, butyl acrylonitrile, hydroxyethyl acrylonitrile, taurate, C₁₀₋₃₀ alkyl acrylonitrile, and 2-acrylamido-2-methylpropane sulfonic acid according to some embodiments. At least one acrylic polymer may be a carbomer that may be polyacrylic acid. At least one acrylic polymer may be a hydrophobically modified carbomer. At least one acrylic polymer is present at a concentration from about 5 wt. % to about 50 wt. %, by weight of a cold process emulsifier composition. At least one acrylic polymer may be present at a concentration of about 5 wt. %, or about 10 wt. %, or about 15 wt. %, or about 20 wt. %, or about 25 wt. %, or about 30 wt. %, or about 35 wt. %, or about 40 wt. %, or about 50 wt. %, in each case, by weight of a cold process emulsifier composition. At least one acrylic polymer may be present at a concentration range comprising from about 5 wt. % to about 10 wt. %, or from about 10 wt. % to about 15 wt. %, or from about 15 wt. % to about 20 wt. %, or from about 20 wt. % to about 25 wt. %, or from about 25 wt. % to about 30 wt. %, or from about 35 wt. % to about 40 wt. %, or from about 40 wt. % to about 45 wt. %, or from about 45 wt. % to about 50 wt. %, in each case, by weight of a cold process emulsifier composition. At least one acrylic polymer may be present at a concentration of about 20 wt. %, by weight of a cold process emulsifier.

In some embodiments, a cold process emulsifier composition may comprise a hydrophobic continuous phase, the hydrophobic continuous phase comprising at least one solvent, wherein the at least one solvent may have a polarity index from about 3 mN/m to about 30 mN/m. At least one solvent may have a polarity index of about 3 mN/m, or of about 5 mN/m, or of about 7.5 mN/m, or of about 10 mN/m, or of about 12.5 mN/m, or of about 15 mN/m, or of about 17.5 mN/m, or of about 20 mN/m, or of about 22.5 mN/m, or of about 25 mN/m, or of 27.5 mN/m, or of about 30 mN/m. At least one solvent may have a polarity index from about 8 mN/m to about 30 mN/m. At least one solvent may have a polarity index of below about 30 mN/m, or below about 25 mN/m, or below about 20 mN/m, or below about 15 mN/m, or below about 10 mN/m, or below about 5 mN/m.

At least one solvent may be selected from the group consisting of ethylhexyl oleate, ethylhexyl olivate, ethyl olivate, ethyl oleate, ethyl hexyl linoleate, ethyl hexyl linolenate, ethyl hexyl palmitate, ethyl hexyl stearate, ethyl hexyl salicylate, ethyl hexyl isononanoate, butyl octyl oleate, butyl octyl linoleate, butyl octyl linolenate, butyl octyl palmitate, butyl octyl stearate, butyl octyl salicylate, butyl octyl isononanoate, isononyl oleate, isononyl linoleate, isononyl linolenate, isononyl palmitate, isononyl stearate, isononyl salicylate, isononyl isononanoate, palmitoleic acid, arachidic acid, 11-eicosenoic acid, behenic acid, erucic acid, lignoceric acid, nervonic acid, oleic acid, linoleic acid, linolenic acid, palmitic acid, stearic acid, jojoba oil. At least one solvent may be esters and ethers of acids selected from the group consisting of palmitoleic acid, arachidic acid, 11-eicosenoic acid, behenic acid, erucic acid, lignoceric acid, nervonic acid, oleic acid, linoleic acid, linolenic acid, palmitic acid, and stearic acid. At least one solvent may be selected from the group consisting of esters of naturally derived acids and short or long chain, linear or branched alcohols. At least one solvent may be ethyl hexyl oleate (i.e., Sensolene®). At least one solvent may be butyl octyl salicylate (i.e., HallBrite® BHB). At least one solvent may be selected from triacylglycerols and/or glycerol ethers formed from glycerol and saturated and/or unsaturated fatty acids selected from the group consisting of oleic acid, linoleic acid, linolenic, palmitic acid, stearic acid, and linolenic acid.

In some embodiments, at least one solvent may be present at a concentration from about 20 wt. % to about 80 wt. %, by weight of a cold process emulsifier composition. According to some embodiments, at least one solvent may be present at a concentration of about 20 wt. %, or about 25 wt. %, or about 30 wt. %, or about 35 wt. %, or about 40 wt. %, or about 45 wt. %, or about 50 wt. %, or about 55 wt. %, or about 60 wt. %, or about 65 wt. %, or about 70 wt. %, or about 75 wt. %, or about 80 wt. %, in each case, by weight of a cold process emulsifier composition. At least one solvent may be present at a concentration from about 40 wt. % to about 45 wt. %, by weight of a cold process emulsifier composition. At least one solvent may be present at a concentration of about 40 wt. %, by weight of a cold process emulsifier composition. At least one solvent may ensure a correct dispersion of at least one acrylic polymer. Ensuring a correct dispersion of at least one acrylic polymer may stabilize a cold process emulsifier composition.

According to some embodiments, a cold process emulsifier composition may comprise a hydrophilic discontinuous phase comprising water. In some embodiments, water may comprise brine, deionized water, distilled water, filtered water, ultra-filtered water, or combinations thereof.

In some embodiments, a cold process emulsifier composition may comprise at least one emulsifier (i.e., emulsifying agent, emulgent). At least one emulsifier may have a hydrophilic-lipophilic balance from about 2 to about 7. In some embodiments, at least one emulsifier may have a hydrophilic-lipophilic balance from about 3 to about 5. At least one emulsifier may have a hydrophilic-lipophilic balance of about 4. At least one emulsifier may be selected from the group consisting of sorbitan olivate, sorbitan oleate, sorbitan laurate, sorbitan stearate, sorbitan linoleate, sorbitan myristate, sorbitan palmitate, sorbitan arachidonate, cetearyl olivate, cetearyl laurate, cetearyl stearate, cetearyl linoleate, cetearyl myristate, cetearyl palmitate, cetearyl arachidonate, polyglyceryl olivate, polyglyceryl laurate, polyglyceryl stearate, polyglyceryl linoleate, polyglyceryl myristate, polyglyceryl palmitate, polyglyceryl arachidonate, sorbitan monooleate (i.e., Span® 80). At least one emulsifier may be selected from the group consisting of sorbitan and polyglyceryl esters of natural fatty acids. In some embodiments, at least one emulsifier may be polyglyceryl olivate. At least one emulsifier may be sorbitan olivate. In some embodiments, an emulsifier with a hydrophilic-lipophilic balance from about 2 to about 7 may be compatible with polar solvents (e.g., water, dimethyl sulfoxide, glycerin, glycols, acetonitrile, dimethylformamide, ethyl acetate, methanol, acetone, ethanol, methanol, chloroform, acetic acid), which may allow the formation of water in oil emulsions. At least one emulsifier may be present at a concentration range from about 1 wt. % to about 20 wt. %, by weight of a cold process emulsifier composition. At least one emulsifier may be present at a concentration of about 1 wt. %, of about 2 wt. %, of about 3 wt. %, of about 4 wt. %, of about 5 wt. %, of about 7.5 wt. %, of about 10 wt. %, of about 12.5 wt. %, of about 15 wt. %, of about 17.5 wt. %, of about 20, in each case, by weight of a cold process emulsifier composition. At least one emulsifier may be present at a concentration range from about 3 wt. % to about 13 wt. %, by weight of a cold process emulsifier composition. At least one emulsifier may be present at a concentration from about 6 wt. % to about 7 wt. %, by weight of a cold process emulsifier composition. Emulsifiers may provide a desirable thermodynamic stability to and stabilize a cold process emulsifier composition. An emulsifier may increase the viscosity of a cold process emulsifier composition, wherein it may prevent the cold process emulsifier composition from separating, degrading, destabilizing, and/or decomposing. Emulsifiers derived from natural sources (e.g., olive fruit) may provide stability to emulsions while desirably not causing contact irritation and/or other side-effects that may result in discontinued use. A naturally derived emulsifier (e.g., polyglyceryl olivate) may allow a composition to be substantially free (e.g., free) of irritation, poor taste, toxicity and/or other side-effects that may result from contact with formulations containing petrochemical based oils and/or harsh surfactants. Specifically, emulsifiers derived from olive oil fatty acids (e.g., polyglyceryl olivate) may provide a greater compatibility with skin in comparison to non-naturally derived and/or non-olive oil derived emulsifiers. Emulsifier derived from natural sources may be more compatible with skin lipids (e.g., human skin lipids or animal skin lipids).

According to some embodiments, a cold process emulsifier composition may comprise at least one inverting agent (e.g., emulsion inverter). An inverting agent may facilitate using a cold process emulsifier composition to make an oil in water emulsion. An inverting agent may facilitate (i.e., speed up formulation) using a cold process emulsifier composition to make a product. An inverting agent may be selected from the group consisting of polyglyceryl olivate and polyglyceryl oleate. An inverting agent may be selected from the group consisting of naturally derived surfactants such as acyl glucosides, acyl glutamates, acyl sarcosinates, and acyl polyglyceryl esters. An inverting agent may be polyglyceryl olivate according to some embodiments. An inverting agent may be present at a concentration from about 1 wt. % to about 10 wt. %, by weight of a cold process emulsifier composition. According to some embodiments, at least one inverting agent may be present at a concentration of about 1 wt %, or about 1.5 wt %, or about 2 wt %, or about 2.5 wt %, or about 3 wt %, or about 3.5 wt %, or about 4 wt %, or about 4.5 wt %, or about 5 wt %, or about 5.5 wt %, or about 6 wt %, or about 6.5 wt %, or about 7 wt %, or about 7.5 wt %, or about 8 wt %, or about 8.5 wt %, or about 9 wt %, or about 9.5 wt %, or about 10 wt %, in each case, by weight of a cold process emulsifier composition. At least one inverting agent may be present at a concentration of about 5.5 wt. %, by weight of a cold process emulsifier composition. At least one inverting agent at a concentration of about 5.5 wt. %, by weight of a cold process emulsifier composition may provide a correct balance between speed of use and stability of the cold process emulsifier composition. At least one inverting agent may have a hydrophilic-lipophilic balance of at least about 10. At least one inverting agent may have a hydrophilic-lipophilic balance from about 8 to about 12. At least one inverting agent may have a hydrophilic-lipophilic balance from about 5 to about 20, or from about 7 to about 17, or from about 9 to about 11. In some embodiments, an inverting agent may invert a water in oil emulsion to an oil in water emulsion. An inverting agent may invert an oil in water emulsion to a water in oil emulsion. An inverting agent may facilitate using a cold process emulsifier composition to make an oil in water emulsion.

A cold process emulsifier composition may have a viscosity from about 1000 centipoise (“cP”) to about 100,000 cP. A viscosity may be measured at a temperature of about 10° C., of about 15° C., or about 20° C., or about 25° C., or about 30° C., or about 35° C., or about 40° C., or about 45° C., or about 50° C. A viscosity may be measured at a temperature from about 15° C. to about 25° C. A viscosity may be measured at a temperature from about 20° C. In some embodiments, a cold process emulsion may have a viscosity of about 1,000 cP, or about 5,000 cP, or about 10,000 cP, or about 15,000 cP, or about 20,000 cP, or about 25,000 cP, or about 30,000 cP, or about 40,000 cP, or about 50,000 cP, or about 60,000 cP, or about 70,000 cP, or about 80,000 cP, or about 90,000 cP, or about 100,000 cP. A cold process emulsifier composition may have a viscosity from about 10,000 cP to about 20,000 cP. A cold process emulsifier composition may have a viscosity from about 10,000 cP to about 20,000 cP at a temperature of about 20° C. In some embodiments, a cold process emulsifier composition may be adjusted to target a specific viscosity and/or viscosity range. Target viscosity and/or viscosity ranges may result in a desired feel and/or flow of cosmetic compositions, personal care product compositions, and/or pharmaceutical compositions. Target viscosity and/or viscosity ranges may result in desirable mechanical properties and/or resistance to separation. Cold process emulsifier compositions may be developed to retain a targeted viscosity at a specific temperature and/or temperature range, which may be used to analyze the stability (i.e., resistance to flocculation, creaming, coalescence, unintentional phase inversion). A target viscosity or viscosity ranges may permit manageability of a cold process emulsifier composition, wherein the cold process emulsifier composition is stable. Manageability may comprises pourability and pumpability. In some embodiments, a cold process emulsifier composition may be adjusted to target a specific viscosity and/or viscosity range. Target viscosity and/or viscosity ranges may result in a desired feel and/or flow of cold process emulsifier compositions. Cold process emulsifier compositions may be developed to retain a targeted viscosity at a specific temperature and/or temperature range, which may be useful for the stability (i.e., resistance to flocculation, creaming, coalescence, unintentional phase inversion).

### Methods of Making Cold Process Emulsifier Compositions

The present disclosure relates, according to some embodiments, to methods of making cold process emulsifier compositions. A method of making a cold process emulsifier composition may comprise (a) combining at least one acrylic polymer with at least one emulsifier and a hydrophobic continuous phase comprising at least one solvent to form an acrylic polymer continuous phase mixture; (b) combining the acrylic polymer continuous phase mixture with a hydrophilic discontinuous phase and at least one basic solution to form a neutralized polyacrylate mixture; and (c) combining optionally at least one inverting agent with the acrylic polymer continuous phase mixture to form the cold process emulsifier composition. A method of making a cold process emulsifier composition may comprise formulating a product with the cold process oil in water emulsion at a temperature from about 20° C. to about 25° C., wherein the product comprises personal care products, pharmaceutical compositions, and cosmetic compositions.

At least one polar solvent may have a polarity index from about 3 mN/m to about 30 mN/m. At least one emulsifier may have a hydrophilic-lipophilic balance from about 2 to about 5. Combining an acrylic polymer continuous phase mixture with a hydrophilic discontinuous phase and at least one basic solution to form a neutralized polyacrylate mixture may be performed at a temperature of less than about less than about 80° C., or less than about 75° C., or less than about 70° C., or less than about 65° C., or less than about 60° C., or less than about 55° C., or less than about 50° C., or less than about 45° C., or less than about 40° C., or less than about 35° C., or less than about 30° C., or less than about 25° C., or less than about 20° C. Neutralization of a polyacrylate mixture may be achieved when a composition comprising at least one acrylic polymer has a pH from about 4 to about 9. Neutralization of a neutralized polyacrylate mixture may be achieved when a composition comprising at least one acrylic polymer has a pH from about 5 to about 8. Neutralization of a neutralized polyacrylate mixture may be achieved when a composition comprising at least one acrylic polymer has a pH from about 6.5 to about 7.5. Neutralization of a neutralized polyacrylate mixture may be achieved when a composition comprising at least one acrylic polymer has a pH of about 7. Neutralization of a neutralized polyacrylate mixture may be achieved when a composition comprising at least one acrylic polymer has a pH of about 4, or of about 4.5, or of about 5, or of about 5.5, or of about 6, or of about 6.5, or of about 7, or of about 7.5, or of about 8, or of about 8.5, or of about 9. A pH of a composition may be measured by diluting the composition to make an about 1 wt. % in water composition, and then measuring the pH of the 1 wt. % in water composition.

In some embodiments, at least one basic solution may comprise water and at least one base selected from the group consisting of lithium hydroxide, sodium hydroxide, potassium hydroxide, cesium hydroxide, calcium hydroxide, ammonium hydroxide, potassium bicarbonate, sodium bicarbonate, calcium bicarbonate, ammonium bicarbonate, potassium carbonate, sodium carbonate, calcium carbonate, ammonium carbonate.

At least one base may be present in at least one basic solution at a concentration from about 5 wt. % to about 70 wt. %, by weight of the at least one basic solution. According to some embodiments, at least one base may be present in at least one basic solution at a concentration of about 5 wt. %, or about 10 wt. %, or about 15 wt. %, or about 20 wt. %, or about 25 wt. %, or about 30 wt. %, or about 35 wt. %, or about 40 wt. %, or about 50 wt. %, or about 55 wt. %, or about 60 wt. %, or about 65 wt. %, or about 70 wt. %, in each case, by weight of the at least one basic solution. At least one base may be present in at least one basic solution at a concentration of about 30 wt. %, by weight of the at least one basic solution. At least one base may be present in at least one basic solution at a concentration from about 5 wt. % to about 10 wt. %, or from about 10 wt. % to about 15 wt. %, or from about 15 wt. % to about 20 wt. %, or from about 20 wt. % to about 25 wt. %, or from about 25 wt. % to about 30 wt. %, or from about 35 wt. % to about 40 wt. %, or from about 40 wt. % to about 45 wt. %, or from about 45 wt. % to about 50 wt. %, or from about 50 wt. % to about 55 wt. %, or from about 55 wt. % to about 60 wt. %, or from about 60 wt. % to about 65 wt. %, or from about 65 wt. % to about 70 wt. %, by weight of the at least one basic solution. At least one base may be present in at least one basic solution at a concentration of about 5 wt. %, about 10 wt. %, about 15 wt. %, about 20 wt. %, about 25 wt. %, about 30 wt. %, about 35 wt. %, about 40 wt. %, about 50 wt. %, about 55 wt. %, about 60 wt. %, about 65 wt. %, about 70 wt. %, in each case, by weight of the at least one basic solution. At least one base may be present in at least one basic solution at a concentration from about 25 wt. % to about 35 wt. %, by weight of the at least one basic solution.

Combining components may involve any desired manner of solid and/or liquid handling means. Methods of making cold process emulsifier compositions may comprise automated and manual means of gas, liquid, semi-solid and/or solid transference and combination. Methods of mixing (i.e., blending) may comprise laminar, turbulent, and/or transitional flow regimes. Methods of mixing may comprise turbine (i.e., impeller), baffle, close-clearance, high shear, static, liquid whistle, and combinations thereof. Mixing may comprise low speed (i.e., frequency of rotation) and high speed mixing. For example, methods of making cold process emulsifier compositions may comprising mixing at a frequency of rotation of about 10 rpm, or at about 100 rpm, or at about 200 rpm, or at about 300 rpm, or at about 400 rpm, or at about 500 rpm, or at about 600 rpm, or at about 700 rpm, or at about 800 rpm, or at about 900 rpm, or at about 1000 rpm, or at about 1,250 rpm, or at about 1,500 rpm, or at about 1,750 rpm, or at about 2,000 rpm, or at about 3,000 rpm, or about or at about 4,000 rpm, or at about 5,000 rpm, or at about 6,000 rpm, or at about 7,000 rpm, or at about 8,000 rpm, or at about 9,000 rpm, or at about 10,000 rpm, or at about 11,000 rpm, or at about 12,000 rpm. In some embodiments, methods of mixing may comprise mixing at a frequency of rotation from about 3,000 rpm to about 10,000 rpm.

The present disclosure relates, according to some embodiments, to methods of preparing cold process emulsifier compositions, wherein the methods may have a temperature of less than about 80° C. A cold process emulsifier composition may comprise a temperature of less than 70° C., or less than about 60° C., or less than about 50° C., or less than about 40° C., or less than about 30° C., or less than about 20° C., or less than about 10° C., or less than about 5° C. Maintaining a low temperature may improve stability of a cold process emulsifier composition, avoiding degradation of the polar solvent. In some embodiments, maintaining lower temperatures may preserve the natural and/or intrinsic benefits of natural ingredients (e.g., naturally derived oils, emulsifiers, and/or inverting agents).

A cold process emulsifier composition may be used to develop, formulate, and/or produce personal care compositions, pharmaceutical compositions, and cosmetic compositions. In some embodiments, a cold process emulsifier composition may be used to develop, formulate, and/or produce personal care compositions, pharmaceutical compositions, and cosmetic compositions without having to heat the cold process emulsifier composition to a temperature above about 100, or above about 95° C., or above about 90° C., or above about 85° C., or above about 80° C., or above about 75° C., or above about 70° C., or above about 65° C., or above about 60° C., or above about 55° C., or above about 50° C., or above about 45° C., or above about 40° C., or above about 35° C., or above about 30° C., or above about 25° C.

### Cold Process Oil in Water Emulsions and Method of Making Cold Process Oil in Water Emulsions From Cold Process Emulsifier Compositions

In some embodiments, the present disclosure relates to cold process oil in water emulsions. In comparison to non cold process oil in water emulsions, a cold process oil in water emulsion may be formed at lower temperatures (e.g., <about 40° C.). High temperatures (e.g., >about 40) may have a less than desirable effect on the environment and other ingredients contained in an emulsion. In comparison to non cold process oil in water emulsions, a cold process oil in water emulsion may be less sensitive to electrolytes. In some embodiments, a cold process oil in water emulsion may be less sensitive to fluctuations in pH. Sensitivity of a cold process oil in water emulsion may comprise being sensitive to a reduction in stability, flocculation, creaming, coalescence, unintentional phase inversion, or a combination thereof. A cold process oil in water emulsion may be better suited for the development of pharmaceutical compositions, cosmetic compositions, and/or personal care product compositions. In some embodiments a cold process oil in water emulsion may be formed by combining and/or mixing a cold process emulsifier composition with at least one hydrophilic continuous phase. Products comprising personal care products, pharmaceutical compositions, and cosmetic compositions may be formulated with a cold process oil in water emulsion at a temperature from about 5° C. to about 40° C.

According to some embodiments, a cold process emulsifier composition comprising at least one neutralized acrylic polymer may swell in the presence of water, or when the cold process emulsifier composition is combined, and/or mixed with an aqueous formulation and/or hydrophilic continuous phase. Combining and/or mixing a cold process emulsifier composition with an aqueous formulation may form an oil in water emulsion, wherein the oil in water emulsion may become more viscous than the cold process emulsifier composition. In some embodiments, combining and/or mixing a cold process emulsifier composition with an aqueous formulation may form an oil in water emulsion, wherein the oil in water emulsion may become less viscous than the cold process emulsifier composition.

A cold process oil in water emulsion may have a viscosity from about 1,000 cP to about 1,000,000 cP. A viscosity may be measured at a temperature of about 10° C., of about 15° C., or about 20° C., or about 25° C., or about 30° C., or about 35° C., or about 40° C., or about 45° C., or about 50° C. A viscosity may be measured at a temperature from about 15° C. to about 25° C. A viscosity may be measured at a temperature from about 20° C. In some embodiments, a cold process emulsion may have a viscosity of about 1,000 cP, or about 10,000 cP, or about 100,000 cP, or about 200,000 cP, or about 300,000 cP, or about 400,000 cP, or about 500,000 cP, or about 600,000 cP, or about 700,000 cP, or about 800,000 cP, or about 900,000 cP, or about 1,000,000 cP. A cold process oil in water emulsion may have a viscosity from about 10,000 cP to about 20,000 cP at a temperature of about 20° C. In some embodiments, a cold process oil in water emulsion may be adjusted to target a specific viscosity and/or viscosity range. Target viscosity and/or viscosity ranges may result in a desired feel and/or flow of cosmetic compositions, personal care product compositions, and/or pharmaceutical compositions. Cold process oil in water emulsions may be developed to retain a targeted viscosity at a specific temperature and/or temperature range, which may be used to analyze the stability (i.e., resistance to flocculation, creaming, coalescence, unintentional phase inversion). A target viscosity or viscosity ranges may permit manageability of a cold process oil in water emulsion, wherein the cold process oil in water is stable. Manageability may comprises pourability and pumpability. In some embodiments, a cold process oil in water emulsion may be adjusted to target a specific viscosity and/or viscosity range. Target viscosity and/or viscosity ranges may result in a desired feel and/or flow of cold process oil in water emulsions. Cold process oil in water emulsions may be developed to retain a targeted viscosity at a specific temperature and/or temperature range, which may be useful for the stability (i.e., resistance to flocculation, creaming, coalescence, unintentional phase inversion).

A cold process oil in water emulsion may be used to develop, formulate, and/or produce personal care compositions, pharmaceutical compositions, and cosmetic compositions. In some embodiments, a cold process oil in water emulsion may be used to develop, formulate, and/or produce personal care compositions, pharmaceutical compositions, and cosmetic compositions without having to heat the cold process oil in water emulsion to a temperature above about 100, or above about 95° C., or above about 90° C., or above about 85° C., or above about 80° C., or above about 75° C., or above about 70° C., or above about 65° C., or above about 60° C., or above about 55° C., or above about 50° C., or above about 45° C., or above about 40° C., or above about 35° C., or above about 30° C., or above about 25° C. A cold process oil in water emulsion may be used to formulate a product at a temperature from about 20° C. to about 25° C. A product may comprise a personal care product (e.g., sun care product, skincare, toiletries), a pharmaceutical, and a cosmetic composition. A cold process oil in water emulsion may remain stable at a temperature of about 5 to about 40° C. for at least about 1 month, or at least about 2 months, or at least about 3 months, or at least about 4 months, or at least about 5 months, or at least about 6 months.

The present disclosure relates, according to some embodiments, to methods of making cold process emulsifier compositions. A method of making a cold process oil in water emulsion may comprise (a) combining an aqueous discontinuous phase with at least one cold process emulsifier component to form a cold process emulsifier composition, the at least one cold process emulsifier component comprising at least one acrylic polymer, wherein the at least one acrylic polymer is neutralized after polymerization by at least one basic solution; a hydrophobic continuous phase comprising at least one solvent, wherein the at least one solvent has a polarity index from about 3 mN/m to about 30 mN/m; at least one emulsifier, wherein the at least one emulsifier has a hydrophilic-lipophilic balance from about 2 to about 5; and at least one inverting agent, wherein the at least one inverting agent has a hydrophilic-lipophilic balance of at least about 10; (b) mixing the cold process emulsifier composition to form a mixed cold process emulsifier composition; and (c) combining the mixed cold process emulsifier composition with at least one hydrophilic continuous phase to form the cold process oil in water emulsion. At least one inverting agent may have a hydrophilic-lipophilic balance from about 8 to about 12.

According to some embodiments, the present disclosure relates to a method of formulating a cold process emulsion product, the method comprising mixing a cold process emulsion composition with at least one emulsion product additive. A cold process emulsion product may comprise personal care products, pharmaceutical compositions, cosmetic compositions, or combinations thereof. At least one emulsion product additive comprises water, glycerin, propylene glycol, butylene glycol, or combinations thereof.

### Personal Care Product Compositions

In some embodiments, the present disclosure relates to a cold process oil in water emulsion suitable for formulating personal care product compositions. According to some embodiments, the present disclosure relates to a cold process oil in water emulsion suitable for formulating personal care product compositions. A personal care product composition may comprise sun care product, sun tan lotion, exfoliants, cleaning pads, colognes, deodorants, lotion, shampoo, conditioners, moisturizers, toothpaste, cleansers, and shaving cream. A personal care product may be formulated to contain at least one cold process oil in water emulsion, wherein formulating the personal care product composition is performed at a temperature from about 5° C. to about 40° C.

### Pharmaceutical Compositions

In some embodiments, the present disclosure relates to a cold process oil in water emulsion suitable for formulating pharmaceutical compositions. According to some embodiments, the present disclosure relates to a cold process oil in water emulsion suitable for formulating pharmaceutical compositions. A pharmaceutical composition may comprise antifungal, antibiotic, vitamin, antihistamine, and antiviral compositions. A pharmaceutical composition may be formulated to contain at least one cold process oil in water emulsion, wherein formulating the pharmaceutical composition is performed at a temperature from about 5° C. to about 40° C.

### Cosmetics Compositions

In some embodiments, the present disclosure relates to cosmetic compositions that may comprise a cold process oil in water emulsion suitable. According to some embodiments, the present disclosure relates to cosmetic compositions that may comprise a cold process oil in water emulsion. A cosmetic composition may comprise a primer, lipstick. lip gloss, a concealer, foundation, concealer, mascara, eye shadow, eye liner, and nail polish. A cosmetic composition may be formulated to contain at least one cold process oil in water emulsion, wherein formulating the cosmetic composition is performed at a temperature from about 5° C. to about 40° C.

As will be understood by those skilled in the art who have the benefit of the instant disclosure, other equivalent or alternative compositions, devices, methods, and systems for cold process emulsions and/or emulsifiers can be envisioned without departing from the description contained herein. Accordingly, the manner of carrying out the disclosure as shown and described is to be construed as illustrative only.

Persons skilled in the art may make various changes in the shape, size, number, substrates, solvents, and/or arrangement of parts without departing from the scope of the instant disclosure. Each disclosed method and method step may be performed in association with any other disclosed method or method step and in any order according to some embodiments. Where the verb “may” appears, it is intended to convey an optional and/or permissive condition, but its use is not intended to suggest any lack of operability unless otherwise indicated. Where open terms such as “having” or “comprising” are used, one of ordinary skill in the art having the benefit of the instant disclosure will appreciate that the disclosed features or steps optionally may be combined with additional features or steps. Such option may not be exercised and, indeed, in some embodiments, disclosed systems, compositions, apparatuses, and/or methods may exclude any other features or steps beyond those disclosed herein. Elements, compositions, devices, systems, methods, and method steps not recited may be included or excluded as desired or required. Persons skilled in the art may make various changes in methods of preparing and using a composition, device, and/or system of the disclosure. For example, a composition, device, and/or system may be prepared and/or used as appropriate for animal and/or human use (e.g., with regard to sanitary, infectivity, safety, toxicity, biometric, and other considerations).

Also, where ranges have been provided, the disclosed endpoints may be treated as exact and/or approximations as desired or demanded by the particular embodiment. Where the endpoints are approximate, the degree of flexibility may vary in proportion to the order of magnitude of the range. For example, on one hand, a range endpoint of about 50 in the context of a range of about 5 to about 50 may include 50.5, but not 52.5 or 55 and, on the other hand, a range endpoint of about 50 in the context of a range of about 0.5 to about 50 may include 55, but not 60 or 75. In addition, it may be desirable, in some embodiments, to mix and match range endpoints. Also, in some embodiments, each figure disclosed (e.g., in one or more of the examples, tables, and/or drawings) may form the basis of a range (e.g., depicted value +/− about 10%, depicted value +/− about 50%, depicted value +/− about 100%) and/or a range endpoint. With respect to the former, a value of 50 depicted in an example, table, and/or drawing may form the basis of a range of, for example, about 45 to about 55, about 25 to about 100, and/or about 0 to about 100. Disclosed percentages are weight percentages except where indicated otherwise.

These equivalents and alternatives along with obvious changes and modifications are intended to be included within the scope of the present disclosure. Accordingly, the foregoing disclosure is intended to be illustrative, but not limiting, of the scope of the disclosure as illustrated by the appended claims.

The title, abstract, background, and headings are provided in compliance with regulations and/or for the convenience of the reader. They include no admissions as to the scope and content of prior art and no limitations applicable to all disclosed embodiments.

### EXAMPLES

Some specific example embodiments of the disclosure may be illustrated by one or more of the examples provided herein.

### Cold Process Emulsifier Composition Preparation:

A 600 ml glass beaker equipped with a stirring apparatus and a thermometer is charged with an acrylic polymer, a hydrophobic polar phase and at least one low HLB emulsifier as indicated below. The mixture is dispersed at 20° C. under vigorous stirring (Ultraturrax® at 3,000 rpm) for 30 minutes. Neutralization is conducted subsequently by adding under anchor stirring, a base solution while maintaining the temperature below about 60° C. Following the neutralization, the composition is cooled to about room temperature (i.e., from about 20° C. to about 25° C.) and optionally added a high HLB emulsion inverter. Tables 1-3 elaborate specific example embodiments and comparative examples of cold process emulsifier compositions.

TABLE 1
Examples 1-5.

| Starting | | | | | |
|---|---|---|---|---|---|
| materials | Ex. 1 | Ex. 2 | Ex. 3 | Ex. 4 | Ex. 5 |
| Acrylic | Acrylates/C10- | Acrylates/C10- | Carbomer | Acrylates/C10- | Acrylates |
| Polymer | 30 Alkyl | 30 Alkyl | 10 g | 30 Alkyl | Copolymer |
| | Acrylate | Acrylate | | Acrylate | 10 g |
| | Crosspolymer | Crosspolymer | | Crosspolymer | |
| | 10 g | 40 g | | 14.5 g | |
| Hydrophobic | Ethylhexyl | Ethylhexyl | Ethylhexyl | Butyloctyl | Ethyl Oleate |
| polar phase | olivate | olivate | olivate | Salicylate | 30 g |
| | (Sensolene ®) | (Sensolene ®) | (Sensolene ®) | (Hallbrite ® | |
| | 30 g | 120 g | 30 g | BHB) 66 g | |
| Low HLB | Sorbitan | Sorbitan | Sorbitan | Polyglyceryl-3 | Sorbitan Oleate |
| emulsifier | Oleate 7 g | Oleate 28 g | Oleate 7 g | Oleate 4.5 g | 5 g |
| Neutralizing | NaOH 30% | NaOH 30% | NaOH 30% | NaOH 30% | NaOH 30% |
| agent | 9.8 g | 39.2 g | 9.8 g | 14.2 g | 9.8 g |
| High HLB | — | Polyglyceryl-5 | — | Polyglyceryl-5 | — |
| emulsifier | | oleate 8 g | | oleate 3.5 g | |
| pH of 1% | 6.9 | 7.1 | 6.2 | 7.2 | 6.2 |
| composition | | | | | |
| in water | | | | | |
| Viscosity, | 2,500 cPs | 5,700 | 7,100 | 2,200 | 9,000 |
| Brookfield | | | | | |

TABLE 2
Examples 6-10.

| Starting | | | | | |
|---|---|---|---|---|---|
| materials | Ex. 6 | Ex. 7 | Ex. 8 | Ex. 9 | Ex. 10 |
| Acrylic | Acrylates/C10- | Acrylates/C10- | Acrylates/C10- | Acrylates/C10- | Acrylates |
| Polymer | 30 Alkyl | 30 Alkyl | 30 Alkyl | 30 Alkyl | Copolymer |
| | Acrylate | Acrylate | Acrylate | Acrylate | 22 g |
| | Crosspolymer | Crosspolymer | Crosspolymer | Crosspolymer | |
| | 15.5 g | 15.5 g | 8.5 g | 8 g | |
| Hydrophobic | Ethylhexyl | Ethylhexyl | Ethylhexyl | Ethylhexyl | Ethylhexyl |
| polar phase | olivate | olivate | olivate | olivate | olivate |
| | (Sensolene ®) | (Sensolene ®) | (Sensolene ®) | (Sensolene ®) | (Sensolene ®) |
| | 31 g | 27.5 g | 31.7 g, | 29.2 g | 42 g |
| | | | Glyceryl | | |
| | | | oleate 25 g | | |
| Low HLB | Polyglyceryl-3 | Sorbitan | Sorbitan | Polyglyceryl-3 | Sorbitan |
| emulsifier | Oleate 4.5 g | Oleate 4.05 g, | Oleate 4.5 g | Oleate 4.5 g | isostearate 2 g, |
| | | Sorbitan | | | Polyglyceryl-4 |
| | | isostearate | | | olivate 6.8 g |
| | | 0.85 g, | | | |
| | | Polyglyceryl-3 | | | |
| | | Oleate 0.45 g | | | |
| Neutralizing | NaOH 30% | NaOH 30% | NaOH 30% | NaOH 10% | NaOH 30% |
| agent | 15.2 g | 15.2 g | 8.3 g | 23.7 g | 21.7 g |
| High HLB | Polyglycery1-5 | Polyglycery1-5 | Polyglycery1-5 | Polyglycery1-5 | Polyglycery1-4 |
| emulsifier | oleate 1.7 g | oleate 3.5 g | oleate 3.5 g | oleate 3.5 g | olivate 5.5 g |
| pH of 1% | 6.8 | 6.9 | 6.3 | 6.8 | 6.8 |
| composition | | | | | |
| in water | | | | | |
| Viscosity, | 32,000 cPs | 82,000 | 1,000 | 30,000 | 11,200 |
| Brookfield | | | | | |

TABLE 3
Comparative Examples 1-5.

| Starting | Comparative | Comparative | Comparative | Comparative | Comparative |
|---|---|---|---|---|---|
| materials | Ex. 1 | Ex. 2 | Ex. 3 | Ex. 4 | Ex. 5 |
| Acrylic | Acrylates/C10- | Acrylates/C10- | Acrylates/C10- | Acrylates/C10- | — |
| Polymer | 30 Alkyl | 30 Alkyl | 30 Alkyl | 30 Alkyl | |
| | Acrylate | Acrylate | Acrylate | Acrylate | |
| | Crosspolymer | Crosspolymer | Crosspolymer | Crosspolymer | |
| | 22 g | 12.5 g | 8.5 g | 8.5 g | |
| Hydrophobic | Ethylhexyl | Ethylhexyl | Ethylhexyl | Ethylhexyl | Ethylhexyl |
| polar phase | olivate | olivate | olivate | olivate | olivate |
| | (Sensolene ®) | (Sensolene ®) | (Sensolene ®) | (Sensolene ®) | (Sensolene ®) |
| | 42 g | 31 g | 56.7 g | 29.8 g | 56.7 g |
| Low HLB | Sorbitan | — | Sorbitan | Sorbitan | Sorbitan |
| emulsifier | isostearate 2 g, | | Oleate 4.5 g | Oleate 4.5 g | Oleate 4.5 g |
| | Polyglyceryl-4 | | | | |
| | olivate 6.8 g | | | | |
| Neutralizing | NaOH 30% | NaOH 30% | Water 8.3 g | Triethanol | Water 8.3 g |
| agent | 21.7 g | 12.2 g | | amine 8.3 g | |
| | Temperature | | | | |
| | >80° C. | | | | |
| High HLB | Polyglyceryl-4 | Polyglyceryl-5 | Polyglyceryl-5 | Polyglyceryl-5 | Polyglyceryl-5 |
| emulsifier | olivate 5.5 g | olivate 1.7 g | oleate 3.5 g | oleate 3.5 g | oleate 3.5 g |
| pH of 1% | 6.8 | not determined | 3.4 | 6.5 | 3.4 |
| composition | | | | | |
| in water | | | | | |
| Viscosity, | Strong gel | Strong dilatant | <1,000 | <1,000 | <1,000 |
| Brookfield | structure, | structure, | | | unstable |
| | >100,000 cP | impossible to | | | emulsion |
| | | work out and | | | |
| | | use | | | |

### Evaluation of Cold Process Emulsifier Compositions:

The Cold process emulsifier composition of the Examples above were employed in the testing compositions described below. Parameters assessed were the viscosity (Brookfield RVT, 24 h, Helipath 92, 20° C., 10 rpm) and the appearance of the composition with results shown in Table 4. Appearance was evaluated using a grading system from 1 to 5, in which appearance grade 1 denotes smooth and homogeneous surface, grade 5 denotes a grainy inhomogeneous surface, with visible gel structures present. From 1 to 3 products are acceptable, 4 to 5 not acceptable.

A 200 ml glass beaker equipped with stirring apparatus is charged with a cold process emulsifier composition and other ingredients as indicated below. A mixture is mixed at room temperature (15-35° C.) under anchor stirring for about 15-30 minutes.

Composition 1: Gel in water

| | |
|---|---|
| Cold process emulsifier composition | 1 wt. % |
| of Examples | |
| Demineralized water | 99 wt. % |
| Preservative | q.s. |

Composition 2: Emulsion

| | |
|---|---|
| Cold process emulsifier composition | 2 wt. % |
| of Examples | |
| Sweet Almond Oil | 20 wt. % |
| Glycerine | 1 wt. % |
| Demineralized water | 88 wt. % |
| Preservative | q.s. |

TABLE 4
Appearance of Examples and Comparative Examples.

| | Composition 1 | | Composition 2 | |
|---|---|---|---|---|
| Example | Viscosity | Appearance | Viscosity | Appearance |
| Ex. 1 | 8,000 | 2 | 18,000 | 2 |
| Ex. 2 | 8,900 | 2 | 20,000 | 1 |
| Ex. 3 | 6,100 | 2 | 30,000 | 2 |
| Ex. 4 | 10,800 | 1 | 22,000 | |
| Ex. 5 | 5,500 | 2 | 123,000 | 2 |
| Ex. 6 | 9,900 | 1 | 31,800 | 1 |
| Ex. 7 | 6,300 | 2 | 30,000 | 1 |
| Ex. 8 | 2,700 | 1 | 17,800 | 1 |
| Ex. 9 | 3,500 | 2 | 28,000 | 2 |
| Ex. 10 | 11,200 | 1 | 32,400 | 1 |
| Comparative | 6,800 | 5 | 24,000 | 5 |
| Ex. 1 | | | | |
| Comparative | <100 | 5 | <100 | 5 |
| Ex. 3 | | | | |
| Comparative | 2,500 | 5 | 15,000 | 5 |
| Ex. 4 | | | | |
| Comparative | <100 | 5 | <100 | 5 |
| Ex. 5 | | | | |
| 1 | | | | |

To demonstrate the possible uses of a cold process emulsifier composition below are reported examples of formulations.

### Preparing Cold Processable Formulations

A 200 ml glass beaker equipped with stirring apparatus is charged with the Cold process emulsifier composition and the other ingredients Phase by Phase as indicated below. The mixture is mixed at room temperature (15-35° C.) under anchor stirring for 15-30 minutes. Viscosity, pH and stability centrifuge test (3,000 rpm, 1 h, <5% separation) tested after 24 h.

Formulation 1: Sun Care

| | | |
|---|---|---|
| PHASE 1 | Water | 61.75 wt. % |
| | WT. Glycerin | 3.00 wt. % |
| | Xanthan gum | 0.20 wt. % |
| | Disodium Ethylenediamine tetraacetic acid | 0.05 wt. % |
| | (EDTA) | |
| PHASE 2 | Ethylhexyl Salicylate | 5.00 wt. % |
| | Octocrylene | 8.00 wt. % |
| | Homosalate | 10.00 wt. % |
| | HallBrite ® BHB | 5.00 wt. % |

HallBrite ® BHB comprises butyloctyl salicylate

| | | |
|---|---|---|
| | Avobenzone | 3.00 wt. % |
| PHASE 3 | VP/Eicosene copolymer | 1.00 wt. % |

VP/Eicosene copolymer comprises 1-eicosene polymer with
1-ethenyl-2-pyrrolidinone)

| | | |
|---|---|---|
| | Cold process emulsifier composition | 2.00 wt. % |
| PHASE 4 | Phenoxyethanol, caprylyl glycol, chlorphenesin | 1.00 wt. % |

Formulation 2: Primer

| | | |
|---|---|---|
| PHASE 1 | Water | 87.60 wt. % |
| | Trisodium ethylendiamine disuccinate | 0.30 wt. % |
| PHASE 2 | Butylated hydroxytoluene | 0.10 wt. % |
| | Olivem ® 1000 (Cetearyl olivate/sorbitan | 3.00 wt. % |
| | olivate) | |
| | Olivem ® 900 (Sorbitan olivate) | 1.00 wt. % |
| PHASE 3 | Methyl methacrylate cross polymer | 2.00 wt. % |
| | Silica (and) CI 77892 (and) CI 7749 | 11.00 wt. % |
| PHASE 4 | Acrylates copolymer | 3.00 wt. % |
| PHASE 5 | Preservative | 1.00 wt. % |
| | Cold process emulsifier composition of | 1.00 wt. % |
| | Ex. 10 | |

Formulation 3: Hair waxy cream

| | | |
|---|---|---|
| PHASE 1 | Water | 84.40 wt. % |
| | Propylene Glycol | 5.00 wt. % |
| | Sorbitol 80% | 5.00 wt. % |
| | Disodium EDTA | 0.05 wt. % |
| PHASE 2 | Olivem ® VS Feel | 1.00 wt. % |

Olivem ® VS Feel comprises Cetearyl alcohol, cetyl palmitate,
sorbitan palmitate, and sorbitan oleate)

| | |
|---|---|
| Cold process emulsifier composition | 2.00 wt. % |
| of Ex. 10 | |
| Biochemica ® Arganshine | 1.00 wt. % |

Biochemica ® Arganshine comprises hydrogenated vegetable oil,
argania spinosa kernel oil, cetearyl alcohol, sweet Almond Oil)

| | | |
|---|---|---|
| | Butylated Hydroxy Toluene (BHT) | 0.05 wt. % |
| PHASE 4 | Preservative | 1.00 wt. % |

Formulation 4: Multiactive Face Serum

| | | |
|---|---|---|
| PHASE 1 | Water | 86.05 wt. % |
| | Chamomilla recutita (matricaria) flower | 5.00 wt. % |
| | water | |
| | Propanediol | 3.00 wt. % |
| | Trisodium Ethylenediamine Disuccinate | 0.60 wt. % |
| PHASE 2 | Cold process emulsifier composition | 1.75 wt. % |
| | of Ex. 10 | |
| | Life Oleobooster ® “” | 0.50 wt. % |

Life Oleobooster ® comprises brassica campestris seed oil,
polyglyceryl-3-diisostearate, stevia rebaudiana extract, and
cistus monspeliensis extract

| | |
|---|---|
| Diam Oleoactif ® | 0.50 wt. % |

Diam Oleoactif ® comprises beta-sitosterol, fridelin, betulin,
quercetine, polyphenols, lauric acid, tocopherols, and vitamin E

| | |
|---|---|
| Epilobium Oleoactif ® | 0.50 wt. % |

Epilobium Oleoactif ® comprises Heliantus Annuus Seed Oil,
and Epilobium Angustifolium extract

| | | |
|---|---|---|
| | Ethylhexyl Isononanoate | 0.75 wt. % |
| | Lecithin, Tocopherol, Ascorbyl Palmitate, | 0.05 wt. % |
| | Citric Acid | |
| PHASE 3 | (Olea Europaea (Olive) Leaf Extract, | 0.30 wt. % |
| | Water) Eurol ® BT | |
| | Preservative | 1.00 wt. % |

What is claimed is:

1. A cold process emulsifier composition comprising:
at least one acrylic polymer neutralized after polymerization by at least one basic solution;
a hydrophobic continuous phase comprising at least one solvent, wherein the at least one solvent has a polarity index from about 3 millinewtons per meter (mN/m) to about 30 mN/m;
a hydrophilic discontinuous phase comprising water;
at least one emulsifier, wherein the at least one emulsifier has a hydrophilic-lipophilic balance from about 2 to about 5; and
at least one inverting agent, wherein the at least one inverting agent has a hydrophilic-lipophilic balance of at least about 10;
wherein the cold process emulsifier composition is configured to form a cold process oil in water emulsion when the cold process emulsifier composition is contacted with a hydrophilic continuous phase comprising at least one aqueous solvent.

2. The cold process emulsifier composition according to claim 1, wherein the at least one basic solution comprises water and at least one base selected from the group consisting of lithium hydroxide, sodium hydroxide, potassium hydroxide, cesium hydroxide, calcium hydroxide, ammonium hydroxide, potassium bicarbonate, sodium bicarbonate, calcium bicarbonate, ammonium bicarbonate, potassium carbonate, sodium carbonate, calcium carbonate, ammonium carbonate.

3. The cold process emulsifier composition according to claim 2, wherein the at least one basic solution has a concentration of the at least one base from about 5 wt. % to about 70 wt. %, by weight of the at least one basic solution.

4. The cold process emulsifier composition according to claim 1, wherein the at least one inverting agent is selected from the group consisting of polyglyceryl olivate and polyglyceryl oleate. An inverting agent may be selected from the group consisting of naturally derived surfactants such as acyl glucosides, acyl glutamates, acyl sarcosinates, and acyl polyglyceryl esters.

5. The cold process emulsifier composition according to claim 1, wherein the at least one inverting agent is polyglyceryl olivate.

6. The cold process emulsifier composition according to claim 1, wherein the at least one inverting agent is present at a concentration from about 1 wt. % to about 10 wt. %, by weight of the cold process emulsifier composition.

7. The cold process emulsifier composition according to claim 1, wherein the at least one acrylic polymer is a homopolymer or copolymer of monomers selected from the group consisting of acrylic acid, methacrylic acid, ethyl acrylic acid, propyl acrylic acid, butyl acrylic acid, methacrylate, ethyl acrylate, propyl acrylate, butyl acrylate, hydroxyethyl acrylate, C₁₀₋₃₀ alkyl acrylate, methyl methacrylate, ethyl methacrylate, propyl methacrylate, butyl methacrylate, hydroxyethyl methacrylate, C₁₀₋₃₀ alkyl methacrylate, acrylonitrile, methyl acrylonitrile, ethyl acrylonitrile, propyl acrylonitrile, butyl acrylonitrile, hydroxyethyl acrylonitrile, taurate, and C₁₀₋₃₀ alkyl acrylonitrile.

8. The cold process emulsifier composition according to claim 1, wherein the at least one acrylic polymer is present at a concentration from about 5 wt. % to about 50 wt. %, by weight of the cold process emulsifier composition.

9. The cold process emulsifier composition according to claim 1, wherein the at least one solvent is selected from the group consisting of ethylhexyl oleate, ethylhexyl olivate, ethyl olivate, ethyl hexyl linoleate, ethyl hexyl linolenate, ethyl hexyl palmitate, ethyl hexyl stearate, and esters and ethers of acids selected from the group consisting of oleic acid, linoleic acid, linolenic acid, palmitic acid, and stearic acid.

10. The cold process emulsifier composition according to claim 1, wherein the at least one solvent is selected from triacylglycerols formed from glycerol and fatty acids selected from the group consisting of oleic acid, linoleic acid, linolenic, palmitic acid, stearic acid, and linolenic acid.

11. The cold process emulsifier composition according to claim 1, wherein the at least one solvent is present at a concentration from about 20 wt. % to about 80 wt. %, by weight of the cold process emulsifier composition.

12. The cold process emulsifier composition according to claim 1, wherein the at least one emulsifier is selected from the group consisting of sorbitan olivate, sorbitan laurate, sorbitan stearate, sorbitan linoleate, sorbitan myristate, sorbitan palmitate, sorbitan arachidonate, cetearyl olivate, cetearyl laurate, cetearyl stearate, cetearyl linoleate, cetearyl myristate, cetearyl palmitate, cetearyl arachidonate, polyglyceryl olivate, polyglyceryl laurate, polyglyceryl stearate, polyglyceryl linoleate, polyglyceryl myristate, polyglyceryl palmitate, and polyglyceryl arachidonate.

13. The cold process emulsifier composition according to claim 1, wherein the at least one emulsifier is present at a concentration from about 1 wt. % to about 20 wt. %, by weight of the cold process emulsifier composition.

14. The cold process emulsifier composition according to claim 1, wherein the at least one aqueous solvent is selected from the group consisting of water, water-based solutions, aqueous-based solutions, and brine.

15. The cold process emulsifier composition according to claim 1, wherein the cold process emulsifier composition has a viscosity of about 5,000 cP to about 25,000 cP.

16. The cold process emulsifier composition according to claim 1, wherein the cold process oil in water emulsion is suitable for formulating personal care products, pharmaceutical compositions, and cosmetic compositions, wherein formulating is performed at a temperature from about 5° C. to about 40° C.

17. A method of making a cold process emulsifier composition, the method comprising:
(a) combining at least one acrylic polymer with at least one emulsifier and a hydrophobic continuous phase comprising at least one solvent to form an acrylic polymer continuous phase mixture;
(b) combining the acrylic polymer continuous phase mixture with a hydrophilic discontinuous phase and at least one basic solution to form a neutralized polyacrylate mixture; and
(c) combining at least one inverting agent with the acrylic polymer continuous phase mixture to form the cold process emulsifier composition.

18. The method of making a cold process emulsifier composition according to claim 17, wherein the at least one acrylic polymer is a homopolymer or copolymer of monomers selected from the group consisting of acrylic acid, methacrylic acid, ethyl acrylic acid, propyl acrylic acid, butyl acrylic acid, methacrylate, ethyl acrylate, propyl acrylate, butyl acrylate, hydroxyethyl acrylate, C₁₀₋₃₀ alkyl acrylate, methyl methacrylate, ethyl methacrylate, propyl methacrylate, butyl methacrylate, hydroxyethyl methacrylate, C₁₀₋₃₀ alkyl methacrylate, acrylonitrile, methyl acrylonitrile, ethyl acrylonitrile, propyl acrylonitrile, butyl acrylonitrile, hydroxyethyl acrylonitrile, taurate, and C₁₀₋₃₀ alkyl acrylonitrile.

19. The method of making a cold process emulsifier composition according to claim 17, wherein the at least one acrylic polymer is present at a concentration from about 5 wt. % to about 50 wt. %, by weight of the cold process emulsifier composition

20. The method of making a cold process emulsifier composition according to claim 17, wherein the at least one solvent is selected from the group consisting of ethylhexyl oleate, ethylhexyl olivate, ethyl olivate, ethyl hexyl linoleate, ethyl hexyl linolenate, ethyl hexyl palmitate, ethyl hexyl stearate, and esters and ethers of acids selected from the group consisting of oleic acid, linoleic acid, linolenic acid, palmitic acid, and stearic acid.

21. The method of making a cold process emulsifier composition according to claim 17, wherein the at least one solvent is selected from triacylglycerols formed from glycerol and fatty acids selected from the group consisting of oleic acid, linoleic acid, linolenic, palmitic acid, stearic acid, and linolenic acid.

22. The method of making a cold process emulsifier composition according to claim 17, wherein the at least one solvent has a polarity index from about 3 millinewtons per meter (mN/m) to about 30 mN/m.

23. The method of making a cold process emulsifier composition according to claim 17, wherein the hydrophilic discontinuous phase comprises water.

24. The method of making a cold process emulsifier composition according to claim 17, wherein the at least one basic solution comprises water and at least one base selected from the group consisting of lithium hydroxide, sodium hydroxide, potassium hydroxide, cesium hydroxide, calcium hydroxide, ammonium hydroxide, potassium bicarbonate, sodium bicarbonate, calcium bicarbonate, ammonium bicarbonate, potassium carbonate, sodium carbonate, calcium carbonate, ammonium carbonate.

25. The method of making a cold process emulsifier composition according to claim 17, wherein the at least one basic solution has a concentration of the at least one base from about 5 wt. % to about 70 wt. %, by weight of the at least one basic solution.

26. The method of making a cold process emulsifier composition according to claim 17, wherein a neutralized polyacrylate mixture has a pH from about 6 to about 8.

27. The method of making a cold process emulsifier composition according to claim 17, wherein the at least one emulsifier has a hydrophilic-lipophilic balance from about 2 to about 5.

28. The method of making a cold process emulsifier composition according to claim 17, wherein the at least one emulsifier is selected from the group consisting of sorbitan olivate, sorbitan laurate, sorbitan stearate, sorbitan linoleate, sorbitan myristate, sorbitan palmitate, sorbitan arachidonate, cetearyl olivate, cetearyl laurate, cetearyl stearate, cetearyl linoleate, cetearyl myristate, cetearyl palmitate, cetearyl arachidonate, polyglyceryl olivate, polyglyceryl laurate, polyglyceryl stearate, polyglyceryl linoleate, polyglyceryl myristate, polyglyceryl palmitate, and polyglyceryl arachidonate.

29. The method of making a cold process emulsifier composition according to claim 17, wherein the at least one inverting agent has a hydrophilic-lipophilic balance of at least about 10.

30. The method of making a cold process emulsifier composition according to claim 17, wherein the at least one inverting agent is polyglyceryl olivate.

31. The method of making a cold process emulsifier composition according to claim 17, further comprising formulating a product with the cold process emulsifier composition at a temperature from about 5° C. to about 40° C., wherein the product comprises personal care products, pharmaceutical compositions, and cosmetic compositions.

32. A method of making a cold process oil in water emulsion, the method comprising:
(a) combining an aqueous discontinuous phase with at least one cold process emulsifier component to form a cold process emulsifier composition, the at least one cold process emulsifier component comprising:
at least one acrylic polymer neutralized after polymerization by at least one basic solution;
a hydrophobic continuous phase comprising at least one solvent, wherein the at least one solvent has a polarity index from about 3 millinewtons per meter (mN/m) to about 30 mN/m;
at least one emulsifier, wherein the at least one emulsifier has a hydrophilic-lipophilic balance from about 2 to about 5; and
at least one inverting agent, wherein the at least one inverting agent has a hydrophilic-lipophilic balance of at least about 10;
(b) mixing the cold process emulsifier composition to form a mixed cold process emulsifier composition; and
(c) combining the mixed cold process emulsifier composition with at least one hydrophilic continuous phase to form the cold process oil in water emulsion.

33. The method of making a cold process oil in water emulsion according to claim 32, wherein the at least one inverting agent is selected from the group consisting of polyglyceryl olivate and polyglyceryl oleate. An inverting agent may be selected from the group consisting of naturally derived surfactants such as acyl glucosides, acyl glutamates, acyl sarcosinates, and acyl polyglyceryl esters.

34. The method of making a cold process oil in water emulsion according to claim 32, wherein combining the aqueous discontinuous phase with the at least one cold process emulsifier component to form the cold process emulsifier composition reaches a concentration of the at least one inverting agent from about 1 wt. % to about 10 wt. %, by weight of the cold process emulsifier composition.

35. The method of making a cold process oil in water emulsion according to claim 32, wherein the at least one acrylic polymer is a homopolymer or copolymer of monomers selected from the group consisting of acrylic acid, methacrylic acid, ethyl acrylic acid, propyl acrylic acid, butyl acrylic acid, methacrylate, ethyl acrylate, propyl acrylate, butyl acrylate, hydroxyethyl acrylate, C₁₀₋₃₀ alkyl acrylate, methyl methacrylate, ethyl methacrylate, propyl methacrylate, butyl methacrylate, hydroxyethyl methacrylate, C₁₀₋₃₀ alkyl methacrylate, acrylonitrile, methyl acrylonitrile, ethyl acrylonitrile, propyl acrylonitrile, butyl acrylonitrile, hydroxyethyl acrylonitrile, taurate, and C₁₀₋₃₀ alkyl acrylonitrile.

36. The method of making a cold process oil in water emulsion according to claim 32, wherein the at least one acrylic polymer is present at a concentration from about 5 wt. % to about 50 wt. %, by weight of the cold process emulsifier composition.

37. The method of making a cold process oil in water emulsion according to claim 32, wherein the at least one solvent is selected from the group consisting of ethylhexyl oleate, ethylhexyl olivate, ethyl olivate, ethyl hexyl linoleate, ethyl hexyl linolenate, ethyl hexyl palmitate, ethyl hexyl stearate, and esters and ethers of acids selected from the group consisting of oleic acid, linoleic acid, linolenic acid, palmitic acid, and stearic acid.

38. The method of making a cold process oil in water emulsion according to claim 32, wherein the at least one solvent is selected from triacylglycerols formed from glycerol and fatty acids selected from the group consisting of oleic acid, linoleic acid, linolenic, palmitic acid, stearic acid, and linolenic acid.

39. The method of making a cold process oil in water emulsion according to claim 32, wherein the at least one solvent is present at a concentration from about 20 wt. % to about 80 wt. %, by weight of the cold process emulsifier composition.

40. The method of making a cold process oil in water emulsion according to claim 32, wherein the at least one emulsifier is selected from the group consisting of sorbitan olivate, sorbitan laurate, sorbitan stearate, sorbitan linoleate, sorbitan myristate, sorbitan palmitate, sorbitan arachidonate, cetearyl olivate, cetearyl laurate, cetearyl stearate, cetearyl linoleate, cetearyl myristate, cetearyl palmitate, cetearyl arachidonate, polyglyceryl olivate, polyglyceryl laurate, polyglyceryl stearate, polyglyceryl linoleate, polyglyceryl myristate, polyglyceryl palmitate, and polyglyceryl arachidonate.

41. The method of making a cold process oil in water emulsion according to claim 32, wherein the at least one emulsifier is present at a concentration from about 1 wt. % to about 20 wt. %, by weight of the cold process emulsifier composition.

42. The method of making a cold process oil in water emulsion according to claim 32, wherein the at least one aqueous solvent is selected from the group consisting of water, water-based solutions, aqueous-based solutions, and brine.

43. The method of making a cold process oil in water emulsion according to claim 32, wherein the cold process emulsifier composition comprises a viscosity of about 5,000 cP to about 25,000 cP.

44. The method of making a cold process oil in water emulsion according to claim 32, further comprising formulating a product with the cold process oil in water emulsion at a temperature from about 5° C. to about 40° C., wherein the product comprises personal care products, pharmaceutical compositions, and cosmetic compositions.

45. A method of formulating a cold process emulsion product, the method comprising mixing a cold process emulsion composition with at least one emulsion product additive, wherein the cold process emulsion composition comprises:
at least one acrylic polymer neutralized after polymerization by at least one basic solution;
a hydrophobic continuous phase comprising at least one solvent, wherein the at least one solvent has a polarity index from about 3 millinewtons per meter (mN/m) to about 30 mN/m;
a hydrophilic discontinuous phase comprising water;
at least one emulsifier, wherein the at least one emulsifier has a hydrophilic-lipophilic balance from about 2 to about 5; and
at least one inverting agent, wherein the at least one inverting agent has a hydrophilic-lipophilic balance of at least about 10.

46. The method of formulating a cold process emulsion product according to claim 45, wherein the cold process emulsion product comprises personal care products, pharmaceutical compositions, cosmetic compositions, or combinations thereof.

47. The method of formulating a cold process emulsion product according to claim 45, wherein the at least one emulsion product additive comprises water, glycerin, propylene glycol, butylene glycol, or combinations thereof.
